(19) United States
(12) Patent Application Publication
(10) Pub. No.: US 2006/0095115 A1
(43) Pub. Date: May 4, 2006

(54) Stent and method of manufacturing same

(76) Inventors: Youssef Bladillah, Montreal (CA); Frank Baylis, Beaconsfield (CA)

Correspondence Address: Louis Tessier, P. O. Box 54029, Mount-Royal, QC, H3P 3H4, CA

(21) Appl. No.: 11/250,446

(22) Filed: Oct. 17, 2005

Related U.S. Application Data
(63) Continuation-in-part of application No. 10/841,816, filed on May 10, 2004.
(60) Provisional application No. 60/619,298, filed on Oct. 15, 2004.

(51) Int. Cl. A61F 2/06

(52) U.S. Cl. 623/1.16; 623/1.24

(57) ABSTRACT

A stent insertable in a body lumen. The stent includes a scaffold having interlinked struts forming a scaffold first section and a scaffold second section, the scaffold being deformable substantially radially between a scaffold retracted configuration and a scaffold expanded configuration. The struts are configured and sized such that the coefficient of radial compressibility of the scaffold second section is greater than the coefficient of radial compressibility of the scaffold first section and the coupling coefficient between radial and longitudinal strains of the scaffold second section is greater than the coupling coefficient between radial and longitudinal strains of the scaffold first section.

The present invention claims priority from Provisional Application Ser. No. 60/619,298 filed on Oct. 15, 2005. This application is also a Continuation-in-Part of U.S. patent application Ser. No. 10/841,816 filed on May 10, 2004.

I hereby claim the benefit under Title 35, United States Code, § 120, of the prior, co-pending United States application listed herinabove and, insofar as the subject matter of each of the claims of this application is not disclosed in the manner provided by the first paragraph of Title 35, United States Codes § 112, I acknowledge the duty to disclose material information as defined in Title 37, Code of Federal Regulations, § 1.56(a), which occurred between the filing date of this application and the national or PCT international filing date of this application Ser. No. 10/841,816, Filed on May 10, 2004.

### FIELD OF THE INVENTION

The present invention relates generally to prosthetic devices. More specifically, the present invention is concerned with a stent.

### BACKGROUND OF THE INVENTION

A stent is a device insertable in a body lumen or body cavity. Stents are used to treat many medical conditions. For example, and non-limitingly, some stents are implanted to open an obstructed or partially obstructed lumen of a body vessel. Other stents include a valve for controlling the flow of a body fluid within a body vessel into which they are implanted. Yet other stents are used in many other medical procedures.

Many stents are insertable percutaneously. These stents are typically inserted in a retracted configuration and subsequently moved through the lumen of various body vessels to a destination where they are deployed.

Specific examples of such stents include a scaffold covered by a sheath. The sheath is typically manufactured separately from the scaffold. Then, the sheath is stitched to the scaffold.

The use of stitches in a stent has some drawbacks. For example, stitches create weaknesses in the sheath. Accordingly, stress concentrations around these weaknesses may tear the sheath. In addition, the stitches provide locations from which undesirable calcifications may grow.

Some stents include a sheath that extends integrally from a scaffold. An example of such a stent is described in U.S. Pat. No. 6,790,237 issued on Sep. 14, 2004, the content of which is incorporated by reference. The stent described in this patent includes a scaffold made out of a wire mesh. Accordingly, if a similar stent were made so that it could be expanded from a retracted configuration to an expanded configuration, the wires would move with respect to each other and would likely stretch and tear the polymer forming the sheath. It this polymer were made resistant to an extent that it would not be torn while such a stent were expanded, this resilience would probably prevent the wires from moving relative to each other, and the stent would therefore not be deployable.

In percutaneously insertable stents including a valve, the valve is typically stitched to the scaffold. Similarly to the stitches used to attach sheaths to scaffolds, these stitches create stress concentrations that may produce tears in the valve while it is in use or when it is deployed. Furthermore, such valves are relatively time-consuming to manufacture and require that specialized personnel be used to stitch the valve to the scaffold. Yet, furthermore, the stitches typically protrude from the stent and therefore increase the compressed size or delivery size of the stent. Also, the stitches reduce the width to which the stent may be expanded as the stitches occupy a portion of the interior volume of the vessel in which the stent is expanded. Thus, such stents may be unsuitable for use in relatively small body vessels.

The stent described in the above-referenced U.S. Pat. No. 6,790,237 includes a valve that extends integrally from the sheath of the stent. However, in the stent described in this Patent, the valve extends completely from the sheath. It would therefore be relatively hard to control the deployment of such a valve during deployment if it were included in a collapsible stent. In addition, in some stents the valve must be positioned inside a passageway defined by the sheath. It is not clear from this Patent how such stents could be manufactured as only the formation of a valve extending from the end of a scaffold is described.

Another problem encountered in expandable stents is that during deployment, a radial expansion causes a longitudinal retraction of the stent. These retractions make the stent relatively difficult to position accurately so that it ends up at the suitable location after deployment is complete. Some stents include sections that are substantially unstrained while they are being deployed. However, these sections have a geometry rendering these stents relatively weak in radial compression.

Against this background, there exists a need in the industry to provide a novel stent.

An object of the present invention is therefore to provide a stent.

### SUMMARY OF THE INVENTION

In a first broad aspect, the invention provides a stent. The stent is insertable in a body lumen. The stent includes a scaffold having interlinked struts forming a scaffold first section and a scaffold second section, the scaffold being deformable substantially radially between a scaffold retracted configuration and a scaffold expanded configuration, wherein the struts are configured and sized such that

when in the scaffold expanded configuration, the diameter of the scaffold first and second sections is respectively larger than the diameter of the scaffold first and second sections in the scaffold retracted configuration;

the coefficient of radial compressibility of the scaffold second section is greater than the coefficient of radial compressibility of the scaffold first section such that upon a substantially similar compressive force being exerted substantially radially on both the scaffold first and second sections, the scaffold first section will deform substantially radially to a lesser extent than the scaffold first section; and

the coupling coefficient between radial and longitudinal strains of the scaffold second section is greater than the coupling coefficient between radial and longitudinal strains of the scaffold first section such that upon a substantially similar substantially radial deformation being effected on both the scaffold first and second sections, the scaffold first section is substantially longitudinally strained to lesser extent than said scaffold second section.

Advantageously, the stent is relatively easy to manufacture and to operate. The stent is also expandable in relatively small vessels without restricting excessively the flow of body fluids within the vessel.

The stent is also relatively easy to position so that it is expanded at a desired location. Furthermore, the relatively large resistance to radial compression of the scaffold second section helps in keeping the body lumen open after the stent has been implanted.

In some embodiments of the invention, a sheath is mounted to the scaffold. In these embodiments, there is only a relatively low risk that the sheath will be torn when the stent is expanded, at least for a portion of the sheath mounted to the scaffold first section.

In some embodiments of the invention, the struts form the perimeter of cells. Sheath cell portions of the sheath extend across the cells. At least one of the cell is configured such that there is substantially no longitudinal strain imparted on the corresponding sheath cell portion as the scaffold moves between the scaffold retracted and expanded configurations.

In some embodiments of the invention, the stent is stent valve. The stent valve includes a stent as described hereinabove to which a valve is mounted. For example, the valve includes three leaflets extending integrally at least in part from the scaffold.

In some embodiments of the invention, the leaflets are made with a polymer. Such leaflets may be relatively thin while being strong enough to function properly as a valve. Thinner leaflets typically result in stent valves that are compressible to smaller diameters when the scaffold is in the scaffold retracted configuration, which may be a desired property to facilitate delivery of the stent.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1, in a perspective view, illustrates a scaffold of a stent according to an embodiment of the present invention;

FIG. 2, in a side elevation view, illustrates the scaffold of FIG. 1 to which a sheath is mounted;

FIG. 3, in a side cross-sectional view taken along the lines<III-III of FIG. 5, illustrates the scaffold of FIG. 1 to which a sheath and valve leaflets are mounted to form a stent valve;

FIG. 4, in a side elevation view, illustrates a mandrel usable to form the valve leaflets of FIG. 3 and the valve leaflets formed therewith;

FIG. 5, in a top plan view, illustrates the valve leaflets of FIG. 5 in a closed configuration;

FIG. 6, in a top plan view, illustrates the valve leaflets of FIG. 5 in an open configuration;

FIG. 7, in a side elevation view, illustrates cells of the scaffold of FIG. 1 in a configuration corresponding to a scaffold expanded configuration;

FIG. 8, in a side elevation view, illustrates the cells of FIG. 7 in a configuration corresponding to a scaffold retracted configuration;

FIG. 9, in a side elevation view, illustrates an alternative stent valve;

FIG. 10, in a top partial cross-sectional view, taken along the line X-X of FIG. 1, illustrates the stent valve of FIG. 3.

FIG. 11A, in a side elevation view, illustrates an alternative strut usable in a scaffold according to an alternative embodiment of the present invention;

FIG. 11B, in a side elevation view, illustrates another alternative strut usable in a scaffold according to another alternative embodiment of the present invention;

FIG. 11C, in a side elevation view, illustrates yet another alternative strut usable in a scaffold according to yet another alternative embodiment of the present invention;

FIG. 11D, in a side elevation view, illustrates yet another alternative strut usable in a scaffold according to yet another alternative embodiment of the present invention;

FIG. 12, in a side elevation view, illustrates a portion of another alternative stent valve;

FIG. 13, in a flowchart, illustrates a method for manufacturing a stent valve in accordance with an embodiment of the present invention.

### DETAILED DESCRIPTION

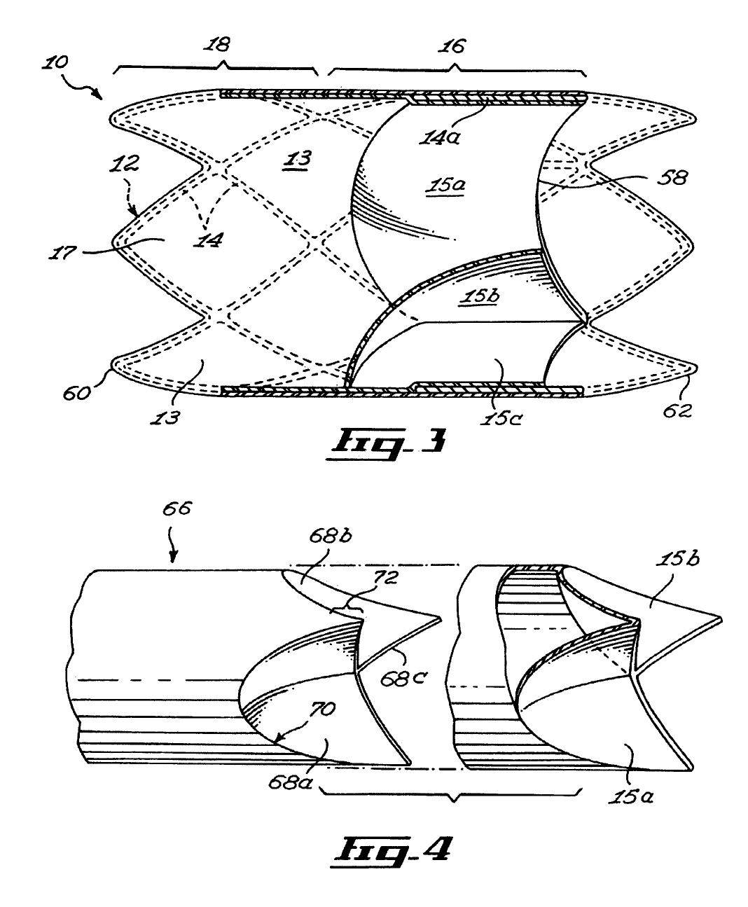

FIG. 3 shows a stent 10 insertable in a body lumen (not shown in the drawings), the stent 10 defining a stent longitudinal axis. The stent includes a scaffold 12, a sheath 13 and valve leaflets 15, 15a and 15b. The sheath 13 and the valve leaflets 15a, 15b and 15c are mounted to the scaffold 12.

The scaffold 12 includes a scaffold passageway 17 that extends substantially longitudinally through the scaffold 12. The valve leaflets 15a, 15b and 15c extend at least partially across a scaffold passageway 17.

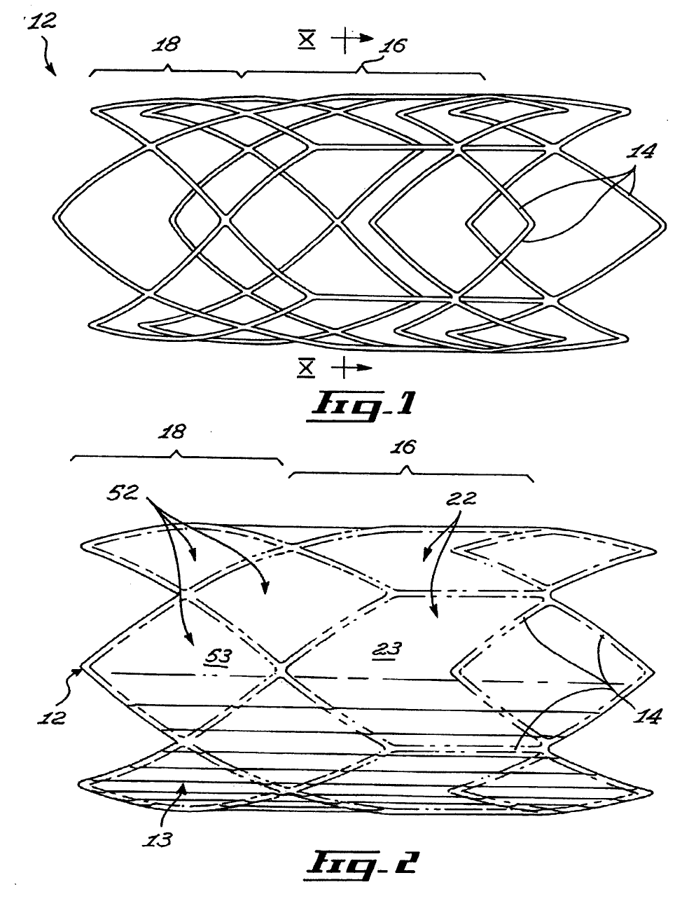

Referring to FIG. 1, the scaffold 12 includes interlinked struts 14 forming the scaffold first section 16 and a scaffold second section 18. The struts 14 are any suitable substantially elongated members interconnected in any suitable manner. For example, the struts 14 each include a substantially elongated metallic member of substantially uniform cross-section, the struts 14 extending integrally from each other. In other embodiments of the invention, struts are secured to each other in any suitable manner, for example through soldering.

The scaffold 12 is deformable substantially radially between a scaffold retracted configuration and a scaffold expanded configuration, shown in FIG. 1. When the scaffold 12 is in the expanded configuration, the diameter of the scaffold first and second sections 16 and 18 is respectively larger than the diameter of the scaffold first and second sections 16 and 18 in the scaffold retracted configuration. The expansion of the stent 10 and of the scaffold 12 is described in further details hereinbelow.

The struts 14 are configured and sized such that the coefficient of radial compressibility of the scaffold second section 18 is greater than the coefficient of radial compressibility of the scaffold first section 16. Therefore, upon a substantially similar compressive force being exerted substantially radially on both the scaffold first and second sections 16 and 18, the scaffold second section 18 will deform substantially radially to a lesser extent than the scaffold first section 16. In other words, the radial strength, i.e. the ability to resist compressive loads, of the scaffold second section 18 is substantially greater than the radial strength of the scaffold first section 16.

Furthermore, the struts 14 are configured and sized such that the coupling coefficient between radial and longitudinal strains of the scaffold second section 18 is greater than the coupling coefficient between radial and longitudinal strains of the scaffold first section 16. Therefore, upon a substantially similar radial deformation being effected on both the scaffold first and second sections 16 and 18, the scaffold first section 16 is substantially longitudinally strained to a lesser extent than the scaffold second section 18. In other words, the effective Poisson's ratio of the scaffold first section 16 is larger than the effective Poisson's ratio of the scaffold second section 18. In yet other words, the relative foreshortening, defined as the reduction in length divided by the length before deformation, of the scaffold first section 16 is substantially smaller than the relative foreshortening of the scaffold second section upon a substantially similar radial deformation being effected on both the scaffold first and second sections 16 and 18.

The reader skilled in the art will readily appreciate that while the stent 10 includes a sheath 13 and valve leaflets 15a, 15b and 15c, it is within the scope of the claimed invention to have a stent that does not include the valve leaflets 15a, 15b and 15c or the sheath 13. Also, it is within the scope of the claimed invention to have a stent that does not include both valve the leaflets 15a, 15b and 15c and the sheath 13. In the latter case, it is within the scope of the claimed invention to have a stent consisting essentially of the scaffold 12.

In some embodiments of the invention, the coefficient of radial compressibility of the scaffold second section 18 is greater than the coefficient of radial compressibility of the scaffold first section 16 when measured in the expanded configuration. However, it is within the scope of the invention to have a coefficient of radial compressibility of the scaffold first and second sections 16 and 18 that satisfy the above-mentioned relationship in any alternative configuration.

In some embodiments of the invention, the coupling coefficient between radial and longitudinal strains of the scaffold second section 18 is greater than the coupling coefficient between radial and longitudinal strains of the scaffold first section 16 when the scaffold 12 is deformed from the retracted configuration to the expanded configuration. However, in the present embodiments of the invention, this property is satisfied for any other suitable deformation.

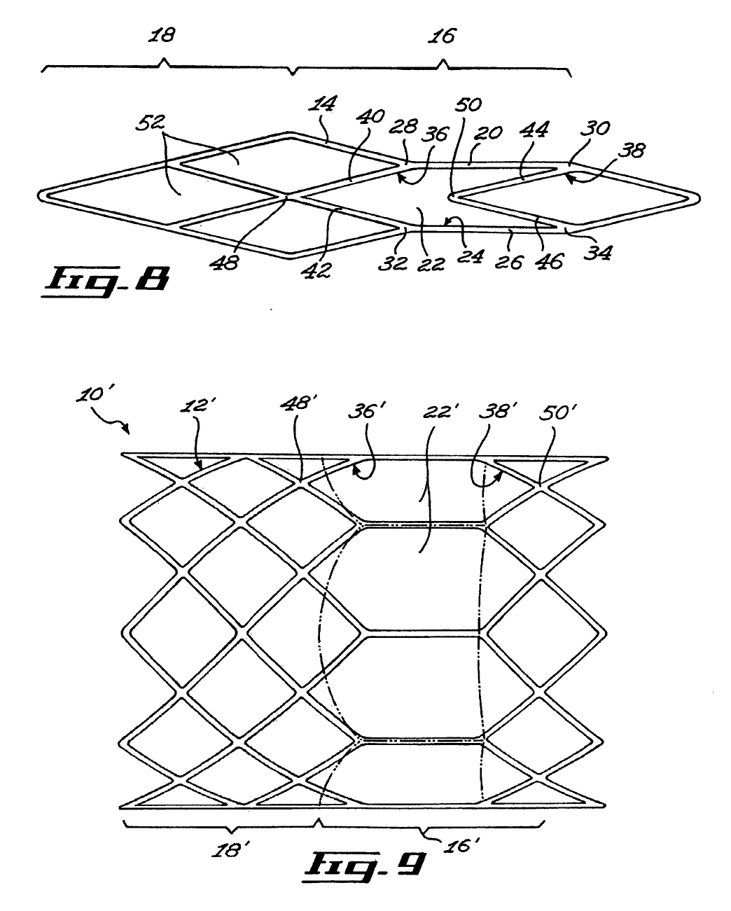

FIGS. 7 and 8 illustrate a few struts 14 of the scaffold 12 when the scaffold 12 is in the expanded configuration (FIG. 7) and when the scaffold 12 is in the retracted configuration (FIG. 8). In the embodiment of the invention shown in these Figures, the struts 14 forming the scaffold first section 16 include at least one longitudinal strut 20 extending in a direction substantially parallel to the stent longitudinal axis.

More specifically, FIGS. 7 and 8 illustrate a detail of the stent 10 wherein the scaffold first section 16 includes a cell 22 having a cell perimeter 24 including two substantially longitudinal struts 20 and 26 that extend in a substantially parallel relationship relative to each other. The longitudinal struts 20 and 26 are interlinked so as to remain in a substantially parallel relationship relative to each other as the stent 10 is deformed between the expanded and the retracted configurations.

The first longitudinal strut 20 defines corresponding longitudinal struts first and second ends 28 and 30. The longitudinal strut 26 defines corresponding longitudinal strut first and second ends 32 and 34. The longitudinal struts 20 and 26 are interconnected substantially adjacent their corresponding first and second ends 28, 30 and 32, 34 by corresponding interconnecting strut arrangements 36 and 38. The interconnecting struts arrangements 36 and 38 have a substantially V-shaped configuration.

To that effect, the interconnecting strut arrangements 36 and 38 define respective pairs of arrangement members 40, 42 and 44, 46 that are pivotally attached together about respective apexes 48 and 50. The arrangement members 36 and 38 are disposed such that the apexes 48 and 50 of the first and second interconnecting struts arrangements 36 and 38 move in the same longitudinal direction as the stent is deformed between the expanded and retracted configurations.

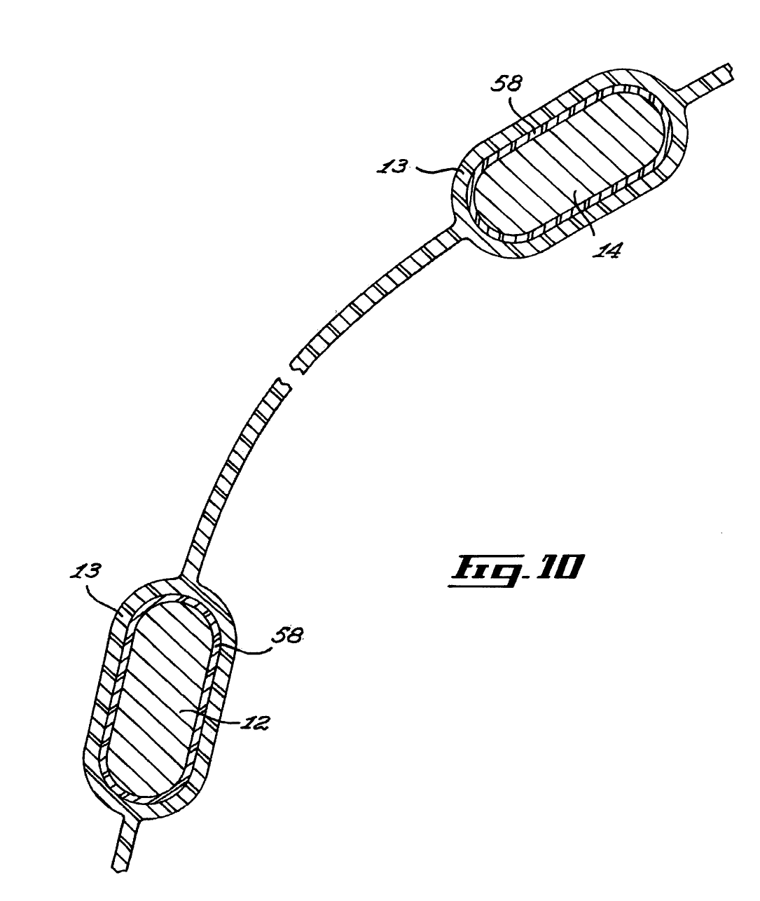

In the embodiment of the invention shown in FIGS. 7 and 8, the apexes 48 and 50 move over the same longitudinal distance and in the same direction as the stent is deformed between the expanded and retracted configurations. In other words, in these embodiments of the invention, the cell 22 is substantially chevron-shaped. However, in alternative embodiments of the invention, these apexes may move in opposite directions. This would be the case with the stent 10′ illustrated in FIG. 9, which is described in further details hereinbelow.

The struts 14 forming the scaffold second section 18 form adjacent second section cells 52. In some embodiments of the invention, the greater resistance to a radial compression of the second section 18 is caused at least in part by a substantially diamond-like shape of the second section cells 52.

Indeed, the reader skilled in the art will readily appreciate that all other factors being equal, the configuration of the cell 52 is substantially less compressible in a circumferential direction than the configuration of the cell 22.

In some embodiments of the invention, the struts 14 forming the scaffold first section 16 include a first material and at least some of the struts 14 forming the scaffold second section 18 include a second material different from the first material. The respective inclusion of the first and second materials in the scaffold first and second sections 16 and 18 causes at least in part the difference in the coefficient of radial compressibility of the scaffold first and second sections. For example, the first material includes nitinol and the second material includes stainless steel. In another example, the first material includes a first stainless steel and the second material includes a second stainless steel having a Young's modulus that differs from the Young's modulus of the first stainless steel. It is also within the scope of the invention to have first and second materials that include any other suitable material.

In other embodiments of the invention, at least some of the struts 14 forming the scaffold first section 16 have a thickness in a substantially radial direction that is substantially different from a thickness in a substantially radial direction of at least some of the struts 14 forming the scaffold second section 18. The difference in thickness between these struts 14 causes at least in part the difference of coefficient of radial compressibility of the scaffold first and second section.

While a specific configuration of the cells forming the scaffold first section 16 have been shown in FIGS. 7 and 8, the reader can readily appreciate that it is within the scope of the invention to have a scaffold first and second sections 16 and 18 including struts 14 forming any other suitable alternative cells.

In some embodiments of the invention, the struts 14 forming the scaffold first section 16 are expandable over a greater range of radial expansion than the struts 14 forming the scaffold second section 18. However, in alternative embodiments of the invention, the scaffold first section 16 is not expandable over a greater range of radial expansion than the scaffold second section 18.

FIG. 2 illustrates an example of a manner of mounting the sheath 13 to the scaffold 12. The valve leaflets 15a, 15b and 15c have been omitted from FIG. 2 for clarity reasons. As better shown in FIG. 10, at least some of the struts 14 are embedded into the sheath 13. The sheath 13 allows radial movement of the struts 14 between the expanded and the retracted configuration with at least some of the struts 14 remaining embedded in the sheath 13 during the radial movement.

In the stent 10, the cells 22 of the scaffold first section 16 and the cells 52 of the scaffold second section 18 each have a respective sheath cell portion 23 and 53 extending thereacross. At least one of the cells 22 is configured such that there is substantially no longitudinal strain imparted on the corresponding sheath cell portion 23 as the scaffold 12 moves between the scaffold retracted and expanded configurations. For example, the substantially chevron-shaped cell 22 has this latter property.

The sheath 13 includes a sheath material. In some embodiments of the invention, the sheath material includes a polymer. For example, the sheath 13 may be formed by a polymer film in which the scaffold 12 is embedded. In other embodiments of the invention, the sheath material includes a biological tissue. In yet other embodiments of the invention, the sheath material is any suitable material.

In some examples of implementation, as seen in FIG. 10, a binding layer 58 is provided between the scaffold 12 and the sheath 13. The binding layer typically coats the scaffold 12.

The binding layer 58 includes a binding material that binds relatively strongly to both the scaffold 12 and the sheath material. Typically, the binding force between the scaffold 12 and the binding material is stronger than the binding force between the scaffold 12 and the sheath material. In these typical embodiments, the binding material improves the binding between the sheath 13 and the structure to which it is mounted, namely the scaffold 12. The resistance of the sheath 13 to tears caused by the exertion of external forces onto the scaffold 12 is therefore improved.

In a specific example of implementation, the scaffold includes a metal and the sheath material includes a sheath polyurethane. In these embodiments, a suitable binding material is a binding polyurethane having different properties. It has been found advantageous in some embodiments of the invention to use a binding polyurethane requiring the application of a larger stress to obtain a predetermined elongation than the stress required to obtain the predetermined elongation with the sheath polyurethane. In very specific examples of implementation, the binding polyurethane requires from about 1.5 to about 10, and sometimes from about 2 to about 3, times larger stresses than the sheath polyurethane to obtain the predetermined strain. An example of such a sheath and binding polyurethane combination is to use polyurethane commercialized under the name Bionate80A as the sheath material and a polyurethane commercialized under the name Bionate55D as the binding material.

It is hypothesized that the increase is binding force between the polyurethane and the scaffold as the polyurethane increases in resistance to elongation is caused by an increase in a number of polar groups in the polyurethane. This increase in the number of polar groups increases the attraction between the polyurethane and the metal through an increase in ionic interactions.

Other non-limiting examples of polymeric sheath materials include polystyrene-b-polyisobutylene-b-polystyrene (SIBS), polyetheretherketone (PEEK), polytetrafluoroethylene (PTFE), and Polynivyl alcohol cryogel (PVAC), among others. These polymeric sheath materials may be usable in cases wherein there is a binding layer 58 or in cases wherein there is no binding layer 58.

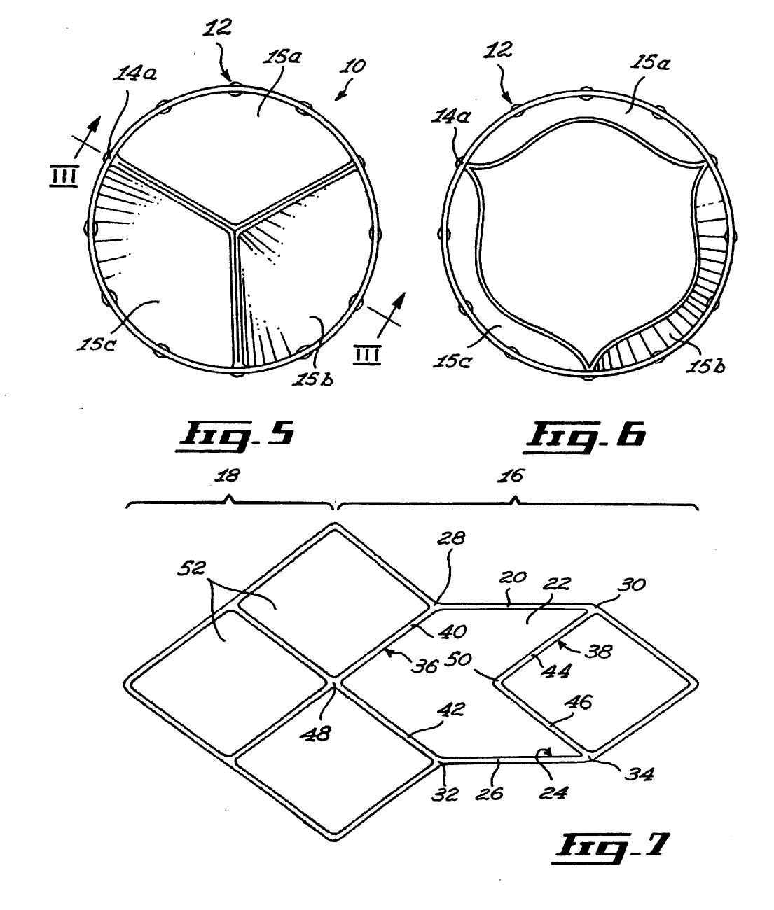

Referring to FIG. 3, the stent 10 defines a stent first end 60 and a longitudinally opposed stent second end 62. The valve leaflets 15a, 15b and 15c are movable between a closed configuration, shown in FIG. 5, and an open configuration, shown in FIG. 6. When the stent 10 is implanted in a body vessel, the flow of a body fluid through scaffold passageway from the stent second end 62 towards the stent first end 60 is substantially prevented in the closed configuration. In the opened configuration, the flow of the fluids between the stent first end 60 and the stent second end 62 is allowed.

The valve leaflet 15a defines a leaflet periphery 58. As seen from FIG. 3, at least a portion of the leaflet periphery 58 extends integrally from at least a portion of at least one of the struts 14. This specific strut is denoted by reference numeral 14a in the drawings. In other words, the valve leaflet 15a originates at least in part from the strut 14a and extends therefrom over a substantially continuous portion of the valve leaflet 15a. Furthermore, at least a portion of the leaflet periphery 58 is substantially parallel to at least a portion of the strut 14a.

This is to be contrasted to “point-like” attachment methods, such as for example the use of stitches to secure a valve leaflet to a scaffold. In other words, the valve leaflet 15a, although it may include a material different from the material forming the strut 14a, extends from the scaffold 12 substantially similarly to a situation wherein a structure made of a single material has a portion that extends directly without discontinuity from another portion thereof. In some embodiments of the invention, aside from the discontinuity formed by the transition in the material composition, there is substantially no discontinuity at the transition from the valve leaflet 15a to the strut 14a. In other embodiments of the invention, there is a molecular attraction between the valve leaflet 15a and the strut 14a, or between the valve leaflet 15a and the binding layer 58, that binds the valve leaflet 15a to the strut 14a, or to the binding layer 58.

As shown in the drawings, the strut 14a extends substantially longitudinally. Therefore, the strut 14a is substantially similar to the struts 20 and 26 shown in FIGS. 7 and 8. In the embodiments of the invention shown in the drawings, as better seen from FIG. 5, two valve leaflets 15a and 15c extend from the strut 14a. These two valve leaflets 15a and 15c intersect only at their leaflet peripheries. In other words, the two valve leaflets 15a and 15c only intersect at the point at which they are attached to the scaffold 12.

As seen from FIG. 3, in some embodiments of the invention, at least a portion of the leaflet periphery 58 extends from the sheath 13. However, in alternative embodiments of the invention, the valve leaflet 15a extends only from the struts 14.

The valve leaflet 15a extends from struts 14 that are embedded into the sheath 13. Therefore, the sheath 13 forms a closed passageway around the valve leaflets 15a, 15b and 15c. This serves, among other purposes, to minimize the paravalvular leaks when the valve leaflets 15a, 15b and 15c are in the closed configuration.

In some embodiments of the invention, the valve leaflets 15a, 15b and 15c include a leaflet material substantially similar to the sheath material. For example, the leaflet material may be the same material as the sheath material. However, in other embodiments of the invention, the sheath material is different from the leaflet material.

The valve leaflets 15b and 15c are substantially similar to the valve leaflet 15a and are therefore not described in further details hereinbelow.

FIG. 13 illustrates an example of a method 100 for manufacturing the stent 10. The method starts at step 102. Then, at step 104, the scaffold 12 is provided. The scaffold may be manufactured in any suitable manner. For example, if the scaffold 12 is a scaffold made of a single material, the scaffold 12 may be cut from a substantially cylindrical shell of the material, for example using laser cutting.

Then, still at step 104 the scaffold 12 is expanded to the expanded configuration if required. Subsequently, at step 106, the scaffold 12 is dipped in the binding material so to form the binding layer 58.

The valve leaflets 15a, 15b and 15c are formed using a mandrel 66, shown in FIG. 4, the mandrel 66 has valve leaflet forming surfaces 68a, 68b and 68c for forming the valve leaflet 15a, only 2 of which are shown in FIG. 4. The valve leaflets forming surface 68a defines a forming surface peripheral edge 70, the forming section peripheral edge 70 including a leaflet-to-strut attachment forming section 72. The valve leaflet forming surfaces 68b and 68c are substantially similar to the valve leaflet forming surface 68a.

At step 108, the valve forming surfaces 68a, 68b and 68c are covered with a stripping substance. The stripping substance is a substance that is soluble in a stripping solvent. The stripping solvent is a fluid into which the stripping substance is soluble but in which the sheath and leaflet materials are substantially insoluble. For example, the stripping substance is an aqueous solution and the stripping substance is Poly (Vinyl Alcohol) (PVOH). In these embodiments, the sheath and leaflet materials may for example include polyurethane, which is not soluble in an aqueous solution. In a specific embodiment of the invention, the stripping substance consists essentially of water.

In some embodiments of the invention, a section of the mandrel 66 that is later dipped in the sheath material is covered with the stripping substance. In yet other embodiments of the invention, the step 108 of covering the valve forming surfaces 68a, 68b and 68c with the stripping substance is omitted.

At step 110, the mandrel 66 is inserted into the scaffold passageway 17. The mandrel 66 is inserted in the scaffold passageway 17 such that the leaflet-to-strut attachment forming section 72 is substantially adjacent and substantially parallel to at least a portion of the strut 14a from which the valve leaflet 15a extends. In embodiments of the invention wherein no valve is formed, no mandrel is inserted in the scaffold passageway. One may then dip-coat the scaffold 12 to obtain a stent having the sheath 13 mounted to the scaffold 12 with not valve formed. This stent would be similar to the view provided on FIG. 2.

At step 112, the valve leaflets 15a, 15b and 15c and the sheath 13 are formed by depositing the leaflet material onto the valve forming surfaces 68a, 68b and 68c and onto the scaffold 12. The step 112 of forming the valve leaflets 15a, 15b and 15c and the sheath 13 may be performed using many techniques.

For example, in some embodiments of the invention, the mandrel and the scaffold 12 are dip-coated. In some embodiments of the invention, the sheath 13 and the valve leaflets 15a, 15b and 15c are dip-coated simultaneously. In other embodiments of the invention, the sheath 13 is first formed without inserting the mandrel 66 into the scaffold passageway 17, for example through dip-coating. Then, in another step, the mandrel 66 is inserted into the scaffold passageway as described hereinabove and the valve leaflets 15a, 15b and 15c are formed. In yet other embodiments of the invention, the valve leaflets 15a, 15b and 15c are formed first and the sheath 13 is formed in another step, for example in another dip-coating step.

In other embodiments of the invention, the polymer film is sprayed onto the scaffold 12 and mandrel 66. In yet other embodiments of the invention, a polymer is molded around the scaffold 12 and onto the valve forming surfaces 68a, 68b and 68c.

In another embodiment of the invention, the polymer film is deposited on the scaffold 12 and valve forming surfaces 68a, 68b and 68c by positioning a first sheet of a polymer so that at least part of this first sheet is in proximity to the scaffold 12 and applying heat to fuse the first sheet to the scaffold 12. The first sheet may be positioned outside the scaffold 12 or inside the scaffold 12. In other embodiments of the invention, the two sheets of polymer are provided inside the scaffold 12 and outside the scaffold 12. These sheets are then fused

Subsequently, at step 114, the mandrel 66 and the stent 10 are dipped into the stripping solvent until at least part of the stripping substance is removed from the mandrel 66. Thereafter, at step 116, the mandrel 66 is removed from the stent 10 and, if required, the valve leaflets 15a, 15b and 15c are separated from each other, for example through laser cutting. The method then ends at step 118. While a specific method for manufacturing the stent 10 has been described hereinabove, it is within the scope of the invention to manufacture the stent 10 in any other suitable manner. Also, while the stent 10 includes the scaffold 12, the valve leaflets 15a, 15b and 15c, and the sheath 13, some of the features described hereinabove may be present in stents that include only a scaffold, in stents having a sheath mounted to a scaffold but having no valve leaflets, to stents including valve leaflets but no sheath, and in any other suitable device.

FIG. 9 illustrates an alternative stent 10′ including an alternative scaffold 12′. The scaffold 12′ is similar to the scaffold 12 except that the scaffold 12′ includes an alternative scaffold first section 16′ including cells 22′ that have a shape different from the shape of the cells 22. More specifically, while the apexes 48 and 50 of the interconnecting arrangements 36 and 38 move in the same direction upon a substantially radial expansion, the cells 22′ deform such that upon a substantially radial expansion, the apexes 48′ and 50′ of alternative interconnecting arrangements 36′ and 38′ move in opposite directions.

An advantage of the cells 22′ relatively to the cells 22 is that the cells 22′ are substantially more rigid radially for similar strut 14 arrangements. An advantage of the cells 22 relatively to the cells 22′ is that a longitudinal strain in the portion of the sheath 13 extending across cells 22 is substantially smaller than a longitudinal strain in the portion of the sheath 13 extending across the cells 22′.

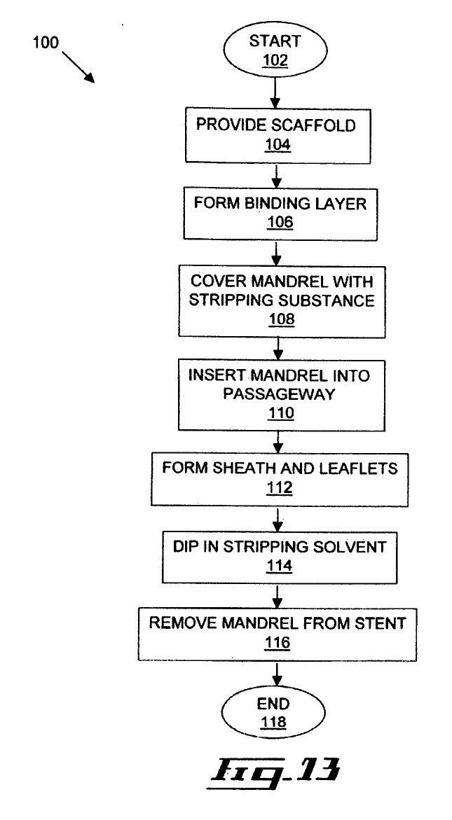

FIG. 12 illustrates a portion of an alternative scaffold 12″ of another alternative stent. The scaffold 12″ is similar to the scaffold 12 except that the scaffold 12″ includes an alternative scaffold first section 16″ including cells 22″ that have a shape different from the shape of the cells 22. More specifically, the cells 22″ have only one longitudinally extending strut 20″. Cells 20″ are formed by having the strut 20″ extending between two apexes of a substantially diamond-shaped cell.

In use, the stent 10 is moved to the retracted configuration. Then, the stent 10 is inserted into a body vessel of a patient and positioned at a suitable location. Then the stent 10 is expanded to the expanded configuration. In some embodiments of the invention, the stent 10 is expanded using a balloon. In other embodiments of the invention, the stent 10 is self-expanding and simply expands once a protective deployment sheath is removed. Techniques for expanding stents are well known in the art and will therefore not be described in further details.

Upon expansion, the sheath encloses the scaffold passageway 17 so as to prevent body fluids circulating in the body vessel to go around the stent once the stent is anchored to the wall of the body vessel.

Since the valve leaflets 15a, 15b and 15c are provided substantially in register with the scaffold first section 16, the valve leaflets 15a, 15b and 15c are relatively easy to position as lateral movements within the scaffold first section 16 are relatively small when the stent 10 is expanded.

Furthermore, substantially no longitudinal strain is induced in the sheath cell portion 23, which reduces the risk of tearing the sheath cell portions 23 during expansion. Since in some embodiments of the invention the sheath 13 is most important around the valve leaflets, the use of cells similar to the cells 23 may be advantageous in these embodiments of the invention.

The sheath 13 and the valve leaflets 15a, 15b and 15c extend integrally from the scaffold 12. This reduces stress concentrations during deployment and operation of the stent 10, and therefore helps in maintaining the structural integrity of the stent 10.

Since the valve leaflets 15a, 15b and 15c extend integrally from the scaffold 12, the expansion of the valve leaflets 15a, 15b and 15c is relatively well controlled as the scaffold 12 may be designed so that it achieves a desired expanded configuration resulting in a predetermined expanded configuration of the valve leaflets 15a, 15b and 15c. Also, the valve leaflets 15a, 15b and 15c do not protrude outside of the sheath 13 and the scaffold 12, which allows to expand the stent 10 so that the valve leaflets 15a, 15b and 15c extend across a relatively large portion of the body vessel. As the performance of a valve is typically dependent on its cross-sectional area, the inventive valve provides relatively good performances during operation.

The relatively rigid construction of the scaffold second section 18 resists radial compressions and therefore allows to have vessels that remain open at a relatively large diameter further to the implantation of the stent 10 in these body vessels.

In some embodiments of the invention, the valve leaflets 15a, 15b and 15c have a substantially uniform thickness. In other embodiments of the invention, the valve leaflets have a substantially non-uniform thickness. For example, and non-limitingly, the valve thickness may be about 150 μm in proximity to the scaffold 12 and about 50 μm at an extremity distal from the scaffold 12. However, other values for the valve leaflet thickness are within the scope of the invention. Having a thicker valve leaflet portion in proximity to the scaffold 12 may be advantageous as is secures relatively strongly the valve leaflets to the scaffold 12. Having a thinner valve leaflet portion away from the scaffold may be advantageous as it reduces a pressure required to open the valve leaflets.

In some embodiments of the invention, the valve leaflets 15a, 15b and 15c extend longitudinally over from about 30% to about 90% of the length of the scaffold 12 in the scaffold expanded configuration. In a specific example of implementation, the valve leaflets 15a, 15b and 15c extend longitudinally over about 70% of the length of the scaffold 12. The longitudinal extension of the valve leaflets 15a, 15b and 15c is determined at least in part by the fluid dynamical properties that are desired for the valve leaflets 15a, 15b and 15c and by the diameter of the scaffold 12 in the expanded configuration.

In some embodiments of the invention, the valve leaflets 15a, 15b and 15c are positioned so that they extend substantially longitudinally centered in the scaffold passageway. This may be advantageous as this positioning typically tends to diminish the influence of end effects cause by sheath 13 on the performance of the valve leaflets 15a, 15b and 15c. For example, a point located midway between the extremities of the valve leaflets 15a, 15b and 15c may be positioned to be distanced from about 0% to about 20% of the length of the scaffold 12 from a location midway between the stent first and second ends 60 and 62.

In some embodiments of the invention, the sheath includes a sheath material and the valve includes a valve material different from said sheath material. In other embodiments of the invention, the sheath material and the valve material are substantially identical.

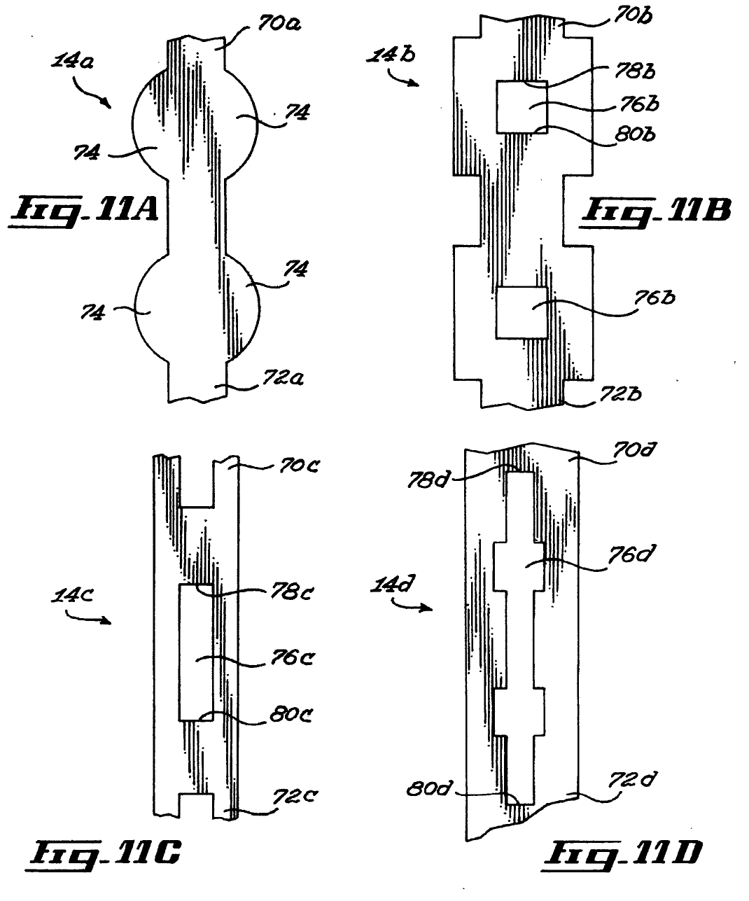

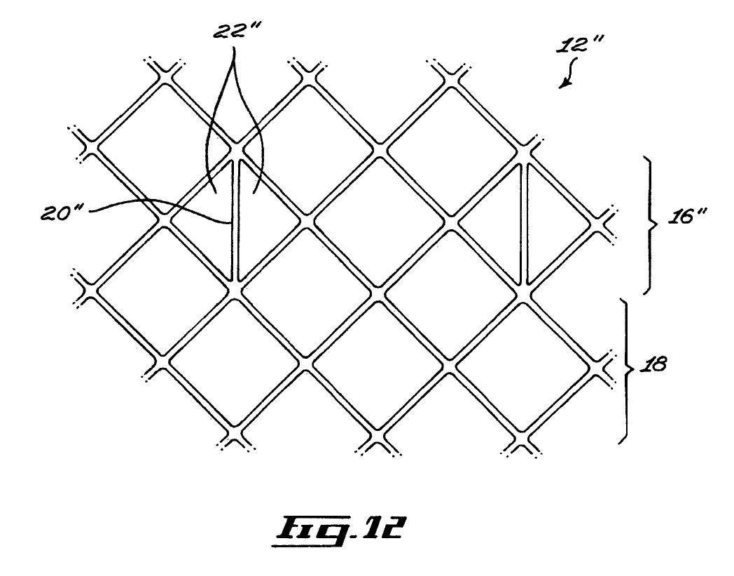

FIGS. 11A, 11B, 11C and 11D illustrate respectively alternative struts 14a, 14b, 14c and 14d. The alternative struts 14a, 14b, 14c and 14d replace at least some of the struts 14 in alternative embodiments of the invention.

The struts 14a, 14b, 14c and 14d are substantially elongated strut. The struts 14a, 14b, 14c and 14d define respective strut longitudinal axes and respective substantially longitudinally opposed strut first and second ends 70a and 72a, 70b and 72b, 70c and 72c, and 70d and 72d.

The struts 14a and 14b have a cross-section in a plane oriented substantially perpendicularly to the strut longitudinal axis that changes in dimensions between the strut first and second ends 70a and 72a, and 70b and 72b. More specifically, the strut 14a defines substantially circumferentially extending strut flanges 74. However it is within the scope of the invention to have struts that have a cross-section that varies in any other suitable manner.

The struts 14b, 14c and 14d each include at least one respective strut aperture 76b, 76c and 76d extending substantially radially from outside the scaffold to inside the scaffold. Some struts aperture have a cross-section in a plane oriented substantially perpendicularly to the strut longitudinal axis that changes in dimensions as a function of a distance from the strut first ends 70b, 70c and 70d. In other words, this cross-section varies in dimensions between substantially longitudinally opposed aperture first and second ends 78b and 80b, 78c and 80c, and 78d and 80d. Valve extends as long as possible and aroung middle of stent

Although the present invention has been described hereinabove by way of preferred embodiments thereof, it can be modified, without departing from the spirit and nature of the subject invention as defined in the appended claims.

What is claimed is:

1. A stent insertable in a body lumen, said stent comprising:
a scaffold including interlinked struts forming a scaffold first section and a scaffold second section, said scaffold being deformable substantially radially between a scaffold retracted configuration and a scaffold expanded configuration, wherein said struts are configured and sized such that
when in said scaffold expanded configuration, the diameter of said scaffold first and second sections is respectively larger than the diameter of said scaffold first and second sections in said scaffold retracted configuration;
the coefficient of radial compressibility of said scaffold second section is greater than the coefficient of radial compressibility of said scaffold first section such that upon a substantially similar compressive force being exerted substantially radially on both said scaffold first and second sections, said scaffold first section will deform substantially radially to a lesser extent than said scaffold first section; and
the coupling coefficient between radial and longitudinal strains of said scaffold second section is greater than the coupling coefficient between radial and longitudinal strains of said scaffold first section such that upon a substantially similar substantially radial deformation being effected on both said scaffold first and second sections, said scaffold first section is substantially longitudinally strained to lesser extent than said scaffold second section.

2. A stent as defined in claim 1, wherein the coefficient the coefficient of radial compressibility of said scaffold second section is greater than the coefficient of radial compressibility of said scaffold first section when said scaffold is in said scaffold expanded configuration, such that upon a substantially similar compressive force being exerted substantially radially on both said scaffold first and second sections when said scaffold is in said scaffold expanded configuration, said scaffold first section will deform substantially radially to a lesser extent than said scaffold first section

3. A stent as defined in claim 1, wherein the coupling coefficient between radial and longitudinal strains of said scaffold second section is greater than the coupling coefficient between radial and longitudinal strains of said scaffold first section such that upon a said scaffold being moved from said scaffold retracted configuration to said scaffold expanded configuration, said scaffold first section is substantially longitudinally strained to lesser extent than said scaffold second section

4. A stent as defined in claim 1, wherein said stent defines a stent longitudinal axis and wherein said struts forming said scaffold first section include a longitudinal strut extending in a direction substantially parallel to said stent longitudinal axis.

5. A stent as defined in claim 4, wherein said struts forming said scaffold first section include a pair of longitudinal struts extending in a substantially parallel relationship relative to each other, said longitudinal struts being interlinked so as to remain in a substantially parallel relationship relative to each other as said stent is deformed between said expanded and retracted configurations.

6. A stent as defined in claim 5, wherein each longitudinal strut defines corresponding longitudinal strut first and second ends, said longitudinal struts being interconnected substantially adjacent their corresponding first and second ends by corresponding interconnecting strut arrangements, said interconnecting strut arrangements having a substantially V-shaped configuration.

7. A stent as defined in claim 6, wherein each of said interconnecting strut arrangements defines a pair of arrangement members pivotally attached together about an apex, said arrangement members being disposed such that said apex of said first and second interconnecting strut arrangements move in the same longitudinal direction as said stent is deformed between said expanded and retracted configurations.

8. A stent as defined in claim 7, wherein said apex of said first and second interconnecting strut arrangements move over the same longitudinal distance direction as said stent is deformed between said expanded and retracted configurations.

9. A stent as defined in claim 6, wherein each of said interconnecting strut arrangements defines a pair of arrangement members pivotally attached together about an apex, said arrangement members being disposed such that said apex of said first and second interconnecting strut arrangements move in opposite longitudinal directions as said stent is deformed between said expanded and retracted configurations.

10. A stent as defined in claim 4, wherein said struts forming said scaffold second section form a cell perimeter of a plurality of adjacent second section cells.

11. A stent as defined in claim 10, wherein at least some of said second section cells are substantially diamond-shaped.

12. A stent as defined in claim 1, further comprising a sheath mounted to said scaffold, wherein said struts forming said scaffold first section form a cell perimeter of a plurality of adjacent first section cells, each of said first section cells having a sheath cell portion extending thereacross, at least one of said cells being configured such that there is substantially no longitudinal strain imparted on the corresponding sheath cell portion as said stent is moved between said stent retracted and expanded configurations.

13. A stent as defined in claim 1, wherein said stent defines a stent first longitudinal end and an opposed stent second longitudinal end, said stent having a stent passageway extending longitudinally thereacross, said stent further comprising at least one valve leaflet extending at least partially across said stent passageway.

14. A stent as defined in claim 13, wherein said valve leaflet is mounted to said scaffold first section.

15. A stent as defined in claim 1, wherein at least some of said struts forming said scaffold first section include a first material and at least some said struts forming said scaffold second sections include a second material different from said first material, the respective inclusion of the first and second materials in the scaffold first and second sections causing at least in part the difference of coefficient of radial compressibility of said scaffold first and second section.

16. A stent as defined in claim 1, wherein at least some of said struts forming said scaffold first section include a first material and at least some said struts forming said scaffold second sections include a second material different from said first material, the respective inclusion of the first and second materials in the scaffold first and second sections causing at least in part the difference of coefficient of radial compressibility of said scaffold first and second section.

17. A stent as defined in claim 16, wherein said first material includes nitinol.

18. A stent as defined in claim 16, wherein said second material includes stainless steel.

19. A stent as defined in claim 16, wherein said first material includes nitinol and said second material includes stainless steel.

20. A stent as defined in claim 16, wherein said first material includes a first stainless steel and said second material includes a second stainless steel, said second stainless steel having a Young's modulus that differs from the Young's modulus of said first stainless steel.

21. A stent as defined in claim 1, wherein at least some of said struts forming said scaffold first section have a thickness in a substantially radial direction that is substantially different from a thickness in a substantially radial direction of at least some of said struts forming said scaffold second section, the difference in thickness between said at least some of said struts forming said scaffold first and second sections causing at least in part the difference of coefficient of radial compressibility of said scaffold first and second section.

22. A stent as defined in claim 1, wherein
at least some of said struts forming said scaffold first section include a first material and at least some said struts forming said scaffold second sections include a second material different from said first material, the respective inclusion of the first and second materials in the scaffold first and second sections causing at least in part the different responses of said scaffold first and second sections to compression and expansion;
said struts forming said scaffold first section form a cell perimeter of a plurality of adjacent first section cells and said struts forming said scaffold second section form a cell perimeter of a plurality of adjacent second section cells, said second section cells having a configuration different from a configuration of said first section cells; and
the difference in the configurations of said first and second section cells causing at least in part the different responses of said scaffold first and second sections to compression and expansion.

23. A stent as defined in claim 1, wherein said struts forming said scaffold first section are expandable over a greater range of radial expansion than said struts forming scaffold second section.

24. A stent as defined in claim 23, wherein said scaffold first and second sections are longitudinally spaced apart, said stent further comprising a a transition section extending between said scaffold first and second sections, said transition section being operatively coupled to said scaffold first and second sections for allowing said scaffold first section to be expandable over a greater range of radial expansion than said scaffold second section.
